(19) United States
(12) Patent Application Publication
(10) Pub. No.: US 2006/0189959 A1
(43) Pub. Date: Aug. 24, 2006

(54) High flow diffusion catheter

(76) Inventor: James A. Schneiter, Lake Forest, IL (US)

Correspondence Address: BRINKS HOFER GILSON & LIONE, P.O. BOX 10395, CHICAGO, IL, 60610, US

(21) Appl. No.: 11/063,198

(22) Filed: Feb. 22, 2005

(51) Int. Cl. A61M 31/00

(52) U.S. Cl. 604/508; 604/523

(57) ABSTRACT

A catheter for use in the withdrawal and/or delivery of a fluid to a patient. The catheter has an elongate tube body including a wall having inner and outer surfaces. The inner surface forms an axial passageway extending through the body in between a proximal end and a distal end. The distal end of the tube body has a rounded bolus tip with a primary port and plurality of secondary ports. The secondary ports extend through the wall at locations in the body spaced from the primary port and from each other. The primary port and the secondary ports are located within a radius of approximately 180 degrees of the circumference of the distal end. A visual indicator is located on at least the proximal end in order to indicate the angular position of the distal end of the catheter.

### FIELD OF THE INVENTION

The present invention relates generally to the field of catheters for use in the delivery and/or removal of a fluid from a patient. More specifically, the present invention relates to a high flow catheter having improved fluid diffusion with a directed fluid flow to minimize vessel damage and trauma, an anti-clog tip to reduce occlusions and an improved surgical implantation system.

### BACKGROUND

In the treatment of many illnesses, it is necessary to infuse medication directly into the bloodstream of a patient. More specifically, many medical procedures require the use of a catheter for the delivery and/or removal of fluids such as medicines, therapeutic agents or blood products from a patient. For example, centrally placed venous access catheters are often used in patients to administer cytotoxic chemotherapy regimes for the treatment of cancer. Because blood flow is greatest in the superior vena cava of the heart, caustic chemotherapy treatments are slowly diffused in the blood stream when they exit the distal end of the central venous catheter. The greater the diffusion of the cytotoxic treatment, the lower the risk of vascular damage/erosion to the superior vena cava of the heart. Also, the faster the diffusion of the cytotoxic treatment into the blood stream, the more effective the therapy for the patient.

The first generation of central venous catheters were external single lumen catheters (“Hickman” and “Broviac”) that are tunneled under the skin and into the subclavian vein with the distal end of the catheter terminating in the superior vena cava of the patient's heart. These external central venous catheters (CVC's) have a polyester cuff around the catheter in order to secure it subcutaneously. These catheters have a number of disadvantages including the need for daily site maintenance and frequent dressing changes where the catheter exits the skin. Past clinical studies have identified significant infection and occlusion rates with these catheters. A high rate of occlusions and infections drastically reduces the effectiveness of the treatment while increasing the potential need for expensive antibiotics and patient hospitalization.

A later generation of central venous catheters were connected to a vascular access port and implanted subcutaneously. These implantable vascular access ports were developed more recently to overcome some of the problems associated with external central venous catheters (daily site maintenance and high rates of infection). While an improvement over external catheters, a review of the clinical studies done on implantable ports over the past 15 years reveals many problems associated with their use.

The most frequent complication associated with ports is occlusion, e.g., withdrawal occlusion or total occlusion. Occlusion is usually the result of a build-up of thrombused blood or drug residuals within the lumen of the catheter or a fibrin sheath formation at the distal end of the catheter. Thrombused blood within a catheter lumen has also been shown to be a nidus for the growth of infectious bacteria. This costly and potentially life threatening complication can be resolved by removing the thrombus formation from the catheter lumen. Thrombused blood within a catheter lumen can only be removed through costly clinical intervention with a lysing agent (urokinase, TPA, etc.) and is not always successful. If catheter patency cannot be restored with a lysing agent, then the patient must undergo a costly and painful surgical procedure to remove the occluded port and associated catheter.

Therefore, there is a need for a catheter that provides for the rapid introduction of medicine with improved diffusion and anti-clog tip that reduces the risk of occlusion.

### BRIEF SUMMARY

The present invention is directed to a catheter that provides for the rapid diffusion of infused medicines or other fluids, improved blood returns and less vascular damage and erosion. The present invention also provides for easier surgical implantation, less turbulence around the ports and improved laminar flow around the outside of the distal end of the catheter while reducing the risk of occlusions and infections and minimizing the shearing effects on any fluid infused/aspirated through the catheter.

According to a first aspect of the invention, a catheter for use in the withdrawal and/or delivery of a fluid to a patient is provided. The catheter has an elongate tube body including a wall having inner and outer surfaces. The inner surface forms an axial passageway extending through the body in between a proximal end and a distal end. The distal end of the tube body has a rounded tip with a primary port and a plurality of secondary ports. The secondary ports extend through the wall at locations in the body spaced from the primary port and from each other. The primary port and the secondary ports are located within a radius of approximately 180 degrees of the circumference of the distal end. A visual indicator is located on at least the proximal end of the catheter in order to indicate the angular position of the distal end thereof. In one embodiment the visual indicator runs along the entire catheter from the distal end to the proximal end.

According to another aspect of the invention, a method for positioning a distal end of a venous catheter within the superior vena cava of a patient in a selected angular position is provided. The method comprises guiding the distal end of the catheter into position within the superior vena cava using a preloaded wire stylet. The distal end of the catheter has a rounded tip with a primary port and plurality of secondary ports. The primary port and the secondary ports are located generally within a top half of the catheter. The method includes using a stylet and a visual indicator located along at least the proximal end of the catheter to adjust the angular position of the distal end of the catheter adjacent a wall in the superior vena cava with the primary port facing away from the wall in the superior vena cava.

### BRIEF DESCRIPTION OF THE DRAWINGS

FIG. 1 is a diagram illustrating the positioning of a catheter within the superior vena cava of a patient according to an embodiment of the present invention.

FIG. 2 is a side view of the catheter embodiment of FIG. 1.

FIG. 3 is a cross-section of the catheter of FIG. 2 taken along the lines 3-3 of FIG. 2 with a stylet shown inserted with the axial passageway of the catheter.

FIG. 4 is a cross-section of a catheter according to another embodiment of the invention for use with a guide wire.

### DETAILED DESCRIPTION OF THE DRAWINGS AND THE PRESENTLY PREFERRED EMBODIMENTS

The preferred embodiments are described herein in the context of catheters, generally. While the present invention is directed to a vascular access catheter it applies equally well to many other types of catheters, including arterial catheters, ascites effusion catheters, pleural cavity effusion catheters, peripherally inserted central catheters (PICC's), mid-line catheters, peritoneal catheters and other catheters useful in a wide range of diverse medical applications that require a catheter for the infusion and/or aspiration of fluids from a patient. The catheter of the present invention is useful for infusion or withdrawal of a wide range of fluids for a patient. As used herein, the term “fluid” should be interpreted broadly to include infused medicines, chemotherapy treatments, blood products, therapeutics, bone marrow transplantation (BMT), antibiotics, total parenteral nutrition (TPA) fluids and to include aspirated venous blood, arterial blood, urine, ascites effusions, pleural cavity effusions and other bodily fluids.

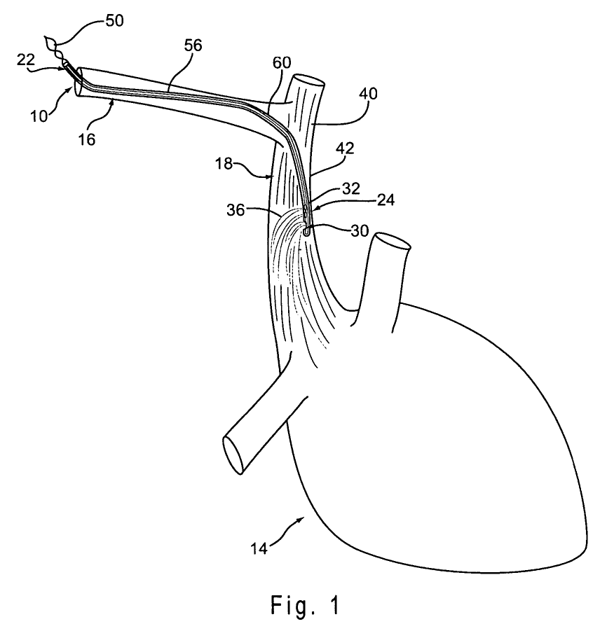

FIG. 1 is an illustration of a catheter 10, according to an embodiment of the present invention that enters the heart 14 of a patient through the right subclavian vein 16 and passes into the superior vena cava 18. The catheter 10 includes a proximal end 22 and distal end 24. The distal end 24 includes a primary port 30 and a plurality of secondary ports 32 that are operable to infuse or withdraw a fluid from the superior vena cava 18 of the heart 14. FIG. 1 illustrates the proper positioning of the distal end 24 such that the infused fluid 36 passes into the bloodstream 40 passing adjacent the primary port 30 and the secondary ports 32. It is an important aspect of the invention that the infused fluid 36 not be directed toward the adjacent wall 42 of the superior vena cava 18. The placement of the distal end 24 of the catheter in this manner helps prevent vascular damage and erosion when cytotoxic chemotherapy treatments are infused into a patient. This orientation of the distal end 24 also helps prevent an occlusion caused by the distal end of a conventional catheter coming into contact with the vessel wall during aspiration.

The catheter 10 may be implanted using a pre-loaded wire stylet 50. While a pre-loaded wire stylet 50 is preferred because of its ease of use, alternate known approaches to implantation include the use of a guide wire or stylet inserted by the implanting surgeon or radiologist. The catheter can be implanted using any and all conventional methodologies with one significant exception. As is well known in the art, the stylet 50 stiffens the catheter 10 during insertion to assist in the proper implantation of the device. The stylet 50 is removed from the catheter 10 upon the proper positioning of the catheter 10. It is important that the stylet 50 and a visual indicator such as implantation stripe 56 be used to control the orientation of the distal end 24 of the catheter 10 such that the primary port 30 and the secondary ports 32 are directed away from the directly adjacent wall 42 of superior vena cava 18. More specifically, the implantation stripe 56 is used by the implanting surgeon or radiologist to insure that the distal end 24 is positioned generally as shown in FIG. 1. By keeping the positioning of the implantation stripe 56 correctly aligned during implantation, the surgeon or radiologist can properly position the distal end 24 so that fluid effusion is directed away from the adjacent wall 42. In the illustrated embodiment, the implantation stripe 56, e.g., an ink stripe, is located on a single side (left side). However, while an implantation stripe 56 is disclosed, it should be recognized that the catheter 10 simply needs to include a visual indicator regardless of shape or size that the surgeon or radiologist may use to properly position the distal end 24 of the catheter 10. The stylet 50 is also useful because a slight downward bend 60 may be placed in the stylet 50 to assist the surgeon or radiologist in directing the distal end 24 downward into the superior vena cava 18 and not upward into the internal jugular vein 40 during the implantation procedure.

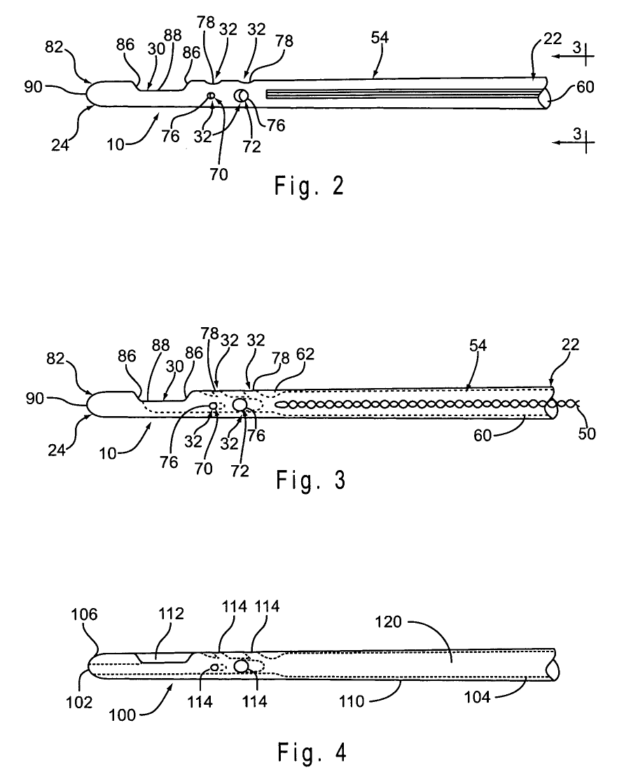

The structure of catheter 10 is best illustrated in FIGS. 2-3. The catheter 10 includes a proximal end 22, a distal end 24 and tube body 54 therebetween. The pre-loaded stylet 50 is shown within an axial passageway 60 extending within the catheter 10. The catheter 10 is useful with many known stylet constructions. The axial passageway 60 includes a narrowed throat portion 62. The narrowed throat portion 62 is adjacent the secondary ports 32 and acts to reduce the diameter of the axial passageway 60 to thereby increase fluid pressure at the secondary ports 32. Increasing the fluid pressure at the secondary ports 32 increases the diffusion rate of the infused fluid and decreases the risk of vessel damage and erosion by directing the infused fluid away from the vessel wall. The plurality of secondary ports 32 are located generally with the top half of the catheter 10. The secondary ports 32 are arranged in two groups 70, 72 of three ports. The two groups 70, 72 extend generally within two parallel planes. Each group 70, 72 of three ports are located about 90 degrees apart around the circumference of the tube body 54. Each group 70, 72 includes two side secondary ports 76 (one shown) and an upper secondary port 78. In the illustrated embodiment, the secondary ports 32 are angled up from the axis of the axial passageway 60. More specifically, in the illustrated embodiment, the secondary ports 32 extend at angle within the range of 20-70 degrees. More specifically, the secondary ports 32 extend at an angle of about 45 degrees from the axis of the axial passageway 60. The secondary ports 32 are preferably circular in shape although other shapes such as oval, oblong, elliptical or the like could be utilized. It should also be recognized that as few as one secondary port and more than six secondary ports may be used with the present invention.

A primary port 30 and anti-clog bolus shaped tip 82 are adjacent the secondary ports 32. The primary port 30 has sloped side walls 86 located between the lateral walls 88. The primary port 30 opens in the same general direction as the upper secondary port 78. The tip 82 includes a rounded bolus-shaped end 90 to assist in implantation and to create a laminar flow around the distal end of the catheter to minimize fluid turbulence, reduce the buildup of drug residuals and/or thrombused blood, and increase patency.

The catheter 10 is preferably fabricated from a resilient, biocompatible and thermoset material such as silicone. Other biocompatible materials such as polyurethane may also be used. Silicone is the preferred material for use with blood and blood products such as encountered during hemodialysis or any other type of long-term venous access. The catheter 10 has a durometer that is preferably within the range of 60 D to 80 D and may be formed from a radiopaque material to facilitate location of the catheter 10 within the patient by use of a fluoroscope or X-ray. The catheter of the present invention is useful in a wide range of French sizes.

FIG. 4 illustrates a catheter 100 according to another embodiment of the invention that operates in essentially the same manner as the catheter 10 with exception that an opening 102 is provided to facilitate implantation using a guide wire rather than a stylet. As in the previous embodiment, the catheter 100 includes a proximal end 104, a distal end 106 and a tube body 110 therebetween. The distal end 106 includes a primary port 112 and a plurality of secondary ports 114 that are operable to infuse or withdraw fluids as described above. The axial passageway extends 120 extends from the proximal end 104 to the opening 102 in the distal end 106.

The embodiments described above and shown herein are illustrative and not restrictive. The scope of the invention is indicated by the claims rather than by the foregoing description and attached drawings. The invention may be embodied in other specific forms without departing from the spirit of the invention. For example, the size, number and shape of the primary and secondary ports may be designed in a manner other than as specifically illustrated in the figures. Also, the present invention may be used with catheters having a wide variety of French sizes and uses. Accordingly, these and any other changes which come within the scope of the claims are intended to be embraced herein.

1. A catheter for use in the withdrawal and/or delivery of a fluid to a patient, comprising:
a) an elongate tube body including a wall having inner and outer surfaces, the inner surface forming an axial passageway extending through the body in between a proximal end and a distal end;
b) the distal end of tube body having a rounded tip with a primary port and a plurality of secondary ports, the secondary ports extending through the wall at locations in the body spaced from the primary port and from each other, the primary port and the secondary ports being located within a radius of approximately 180 degrees of the circumference of the distal end;
c) a visual indicator located on at least the distal end in order to indicate the angular position of the distal end of the catheter.

2. The catheter of claim 1 wherein the visual indicator is a stripe located along the tube body.

3. The catheter of claim 2 wherein the stripe is located along a left side of the tube body.

4. The catheter of claim 2 wherein at least one of the secondary ports has a passageway that forms an angle greater than 20 degrees with respect to an axis of the axial passageway.

5. The catheter of claim 3 wherein at least one of the secondary ports forms an angle substantially equal to 45 degrees with respect to the axis of the axial passageway.

6. The catheter of claim 4 wherein there are at least two secondary ports.

7. The catheter of claim 5 wherein there are at least six secondary ports arranged in a first group and a second group of three secondary ports.

8. The catheter of claim 7 wherein the first group of secondary ports and the second group of secondary ports are formed in two parallel planes.

9. The catheter of claim 6 further comprising a pre-loaded stylet.

10. A catheter for use in the withdrawal and/or delivery of a fluid to a patient, comprising:
a) an elongate tube body including a wall having inner and outer surfaces, the inner surface forming an axial passageway extending through the body in between a proximal end and a distal end, the axial passageway having a narrowed throat portion adjacent the distal end;
b) the distal end of the tube body having a rounded bolus tip with a primary port and a plurality of secondary ports, the secondary ports extending through the wall at locations in the body spaced from the primary port and from each other, the primary port and the secondary ports being located within a radius of approximately 180 degrees of the circumference of the distal end;
c) a visual indicator located on at least the distal end in order to indicate the angular position of the distal end of the catheter.

11. The catheter of claim 10 wherein the visual indicator is a stripe located along the tube body.

12. The catheter of claim 11 wherein the stripe is located along a left side of the tube body.

13. The catheter of claim 12 wherein at least one of the secondary ports has a passageway that forms an angle greater than 20 degrees with respect to an axis of the axial passageway.

14. The catheter of claim 13 wherein there are at least six secondary ports.

15. The catheter of claim 14 wherein the secondary ports are arranged in three groups of two laterally adjacent ports.

16. The catheter of claim 15 wherein the two laterally adjacent ports are co-planer.

17. A method for positioning a distal end of a venous catheter within the superior vena cava of a patient in a selected angular position, the method comprising:
a) guiding a distal end of the catheter to position within the superior vena cava using a preloaded wire stylet, the distal end of the catheter having a rounded tip with a primary port and a plurality of secondary ports, the primary port and the secondary ports located generally within a top half of the catheter; and
b) using the stylet and a visual indicator located along at least the distal end of the catheter to adjust the angular position of the distal end of the catheter adjacent a wall in the superior vena cava with the primary port facing away from the wall in the superior vena cava.

18. The method of claim 17 further comprising the step of removing the stylet.

19. The method of claim 18 wherein the step of using the stylet and the visual indicator includes manipulating the stylet in order to achieve the desired angular position for the distal end of the catheter.

20. The method of claim 19 wherein the step of guiding the distal end includes using the stylet to push the distal end of the catheter into the proper position within the superior vena cava.
